(19) United States
(12) Patent Application Publication
(10) Pub. No.: US 2024/0103017 A1
(43) Pub. Date: Mar. 28, 2024

(54) SERUM PROTEIN BIOMARKER PANEL FOR IDIOPATHIC PULMONARY FIBROSIS

(71) Applicants: Bethany MOORE, Ann Arbor, MI (US); Children's Hospital Medical Center, Cincinnati, OH (US); University of Michigan, Ann Arbor, MI (US)

(72) Inventors: William D. Hardie, Lebanon, OH (US); Assem Ziady, Newport, KY (US); Bethany Moore, Ann Arbor, MI (US)

(21) Appl. No.: 17/766,556

(22) PCT Filed: Oct. 8, 2020
(86) PCT No.: PCT/US20/54664

§ 371 Date: Apr. 5, 2022

Related U.S. Application Data
(60) Provisional application No. 63/024,139, filed on May 13, 2020.
(60) Provisional application No. 62/912,228, filed on Oct. 8, 2019.

(51) Int. Cl. G01N 33/68

(52) U.S. Cl. CPC G01N 33/6893; G01N 2800/12; G01N 2800/52

(57) ABSTRACT

The instant disclosure relates to methods for assessing pulmonary fibrosis disorder disease status in an individual in need thereof. One aspect of the disclosed methods may comprise: detecting a level of one or more biomarkers in a biological sample obtained from an individual, comparing the level of the one or more biomarkers to that of a control value corresponding to the one or more biomarkers, characterizing the disease status in the individual based on the level of the one or more biomarkers as compared to that of a relevant control value; and administering a treatment to said individual based on the assessment of the one or more biomarker levels.

### CROSS-REFERENCE TO RELATED APPLICATIONS

This application claims priority to and benefit of U.S. 62/912,228, filed on Oct. 8, 2019, entitled “Serum Protein Biomarker Panel that Predicts the Development and Resolution of Idiopathic Pulmonary Fibrosis” and U.S. 63/024,139 filed May 13 2020, entitled “Serum Protein Biomarker Panel the Predicts the Development and Resolution of Idiopathic Pulmonary Fibrosis, the contents of each are incorporated in their entirety for all purposes.

### BACKGROUND

Lung fibrosis complicates many interstitial lung diseases (ILD) including systemic connective tissue diseases, childhood interstitial lung disease syndrome, and in response to many types of lung injury¹. The natural history is often inconsistent and unpredictable, and in some specific disorders, such as idiopathic pulmonary fibrosis (IPF), inexorably progressive. This variability in the rate and severity of disease progression makes prognostication for individual patients challenging and creates significant barriers to efficient drug development. Validation of sensitive, reproducible, and objective biomarkers that accurately tracks disease, the fibrotic burden, and response to therapy would be of enormous benefit to clinicians as well as clinical researchers. The current standard of care for diagnosing and monitoring pulmonary fibrosis includes pulmonary function testing (PFTs), imaging by high-resolution computed tomography (HRCT), and surgical lung biopsy. PFTs, specifically spirometry, plethysmography and diffusion, measure lung volumes, airflow and gas uptake and are the widely used for their relative ease in performance, high safety profile, low cost and rapid results. However, PFTs are nonspecific for identifying the underlying pathology. Further, longitudinal monitoring with PFTs is imprecise due to the high variability of endpoints. For instance. Forced vital capacity (FVC), which is often the primary endpoint in IPF clinical trials, varies up to 11% week to week in normal subjects and up to 20% in patients with emphysema^{2,3}. With this variability, PFTs must be obtained and followed over extended periods to confidently follow disease course.

HRCT of the chest is routinely performed in patients with suspected fibrotic lung disease and the presence of typical clinical and radiographic features is sufficient to allow a confident diagnosis of fibrosis in more than 50% of suspected cases. While HRCT provides useful diagnostic information its prognostic value to track disease has several limitations including significant interobserver variability among radiologists in determining the extent of disease.⁴ Another restraint is defining small changes in the fibrotic burden when performing serial studies, especially among individual patients with extensive disease at the time of diagnosis. Further, HRCT has not been shown to reliably identify treatment failure early in trials of therapy.⁵

Surgical lung biopsy is the gold standard for diagnosis pulmonary fibrosis. However, the risk of performing a lung biopsy may be impracticable especially in patients with advanced disease or comorbidities. With an in-hospital mortality of 4.6%, surgical lung biopsies cannot be performed longitudinally to monitor fibrosis progression.⁶

Taken as a whole, currently there are no rapid predictors of disease progression for pulmonary fibrosis disorders. The discovery and development of pulmonary fibrosis-specific biomarkers for use as diagnostic adjuncts or measures of disease activity or treatment response remains a critical unmet need. Newly discovered biomarkers of human disease may reflect disease pathogenesis, change with intervention, and/or offer diagnostic or prognostic value beyond current measures.⁷

The foregoing disclosure addresses one or more of the aforementioned needs in the art.

### BRIEF SUMMARY

The instant disclosure relates to methods for assessing pulmonary fibrosis disorder disease status in an individual in need thereof. One aspect of the disclosed methods may comprise: detecting a level of one or more biomarkers in a biological sample obtained from an individual, comparing the level of the one or more biomarkers to that of a control value corresponding to the one or more biomarkers, characterizing the disease status in the individual based on the level of the one or more biomarkers as compared to that of a relevant control value; and administering a treatment to said individual based on the assessment of the one or more biomarker levels.

### BRIEF DESCRIPTION OF THE DRAWINGS

This application file may contain at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. ELISA confirmation of markers of IPF identified in model mouse plasma by MS proteomics. Protein markers in plasma that significantly changed in the IPPF condition were identified by discovery proteomics and validated by ELISA in mouse and human plasma. Conditions were compared by ANOVA with Dunnett's multiple comparisons correction.

FIG. 2. ELISA confirmation of Ferritin identified by MS proteomics as a marker of IPF resolution. Conditions were compared by ANOVA with Dunnett's multiple comparisons correction *p<0.05; **p<0.01.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

As used herein, the term “measuring” refers to methods which include one or more of detecting the presence or absence of a biomarker in a sample, quantifying the amount of marker(s) in the sample, with or without reference to a control value. Measuring/analyzing/quantifying the level of a biomarker provided herein, may be performed using methods know in the art. For example, methods to determine the level of a biomarker include, but are not limited to, PCR, microarray assays, immunoblots, northern blots, ELISA, fluorescence-based methods (immunofluorescence, FACS), mass spectrometry, and the like.

As used herein, the term “expression” refers to the transcription and stable accumulation of sense (mRNA) or antisense RNA derived from a nucleic acid fragment or fragments. Expression also refers to the translation of mRNA into a polypeptide. Biomarker expression may be one or more of tissue-specific, global, or systemic.

As used herein, “biological sample” may refer to any biological sample from an individual, and may include, for example, blood, serum, plasma, sperm, urine, mucous, tissue biopsy, organ biopsy, synovial fluid, urine, bile fluid, cerebrospinal fluid, saliva, mucosal secretion, effusion, sweat and combinations thereof. In some aspects, the biological sample may be diluted with a suitable diluent before detecting the level of a biomarker in the sample.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Determining the expression of a disclosed biomarker may include assessing gene product or protein abundance. Protein abundance reflects gene expression profiles, which may be determined, by methods known in the art, such as, but not limited to Western blot analysis, RIA, ELISA, HPLC, functional assays, such as enzymatic assays, as applicable, and others. An expression profile may be determined by a change in mRNA levels, surface expression, secretion, or other partitioning of a polypeptide.

“Solid support” refers to a solid material which can be derivatized with, or otherwise attached to, a capture reagent. Exemplary solid supports include probes, microtiter plates and chromatographic resins.

As used herein, “changed expression” or “change in level or “changed level” refer to a change in the level or amount of a biomarker relative to the level or activity of the biomarker in a standard. A change in level may refer to between a 10 to about a 1000% change in biomarker levels in a biological sample. The change in a biological maker level taken from an individual may be 1-10%, 11-20%, 21-30%, 31-40%, 41-50%, 51-60%, 61-70%, 71-80%, 81-90%, 91-150%, 151-1000% elevated or reduced as compared to the level(s) of that biomarkertaken from a normal subject (standard).

In one aspect, disclosed herein are methods for assessing pulmonary fibrosis disorder disease status in an individual in need thereof. The method may comprise
a. detecting a level of one or more biomarkers in a biological sample obtained from said individual;
b. comparing the level to that of a control value;
c. characterizing the disease status in said individual based on the level detected in the individual as compared to that of a control value; and
d. administering a treatment to said individual, wherein the treatment selection is based on the characterization step.

In one aspect, the biomarker may be selected from one or more of selected from Ephrin B2, Epidermal Growth Factor, Ferritin-heavy chain, Apotransferrin, Phosphoinositide 3-kinase, Mdm2-binding protein isoform X3, Tubulin polyglutamylase TTLL13 isoform X7, Heparin sulfate glucosamine 3-O-sulfotransferase 2, pregnancy zone protein, serotransferrin, alpha-2-macroglobulin, immunoglobulin lambda-like polypeptide 5, ceruloplasmin, alpha-1-antitrypsin precursor, complement C3 preproprotein, coiled-coil domain-containing protein 144A, cytochrome P450 3A43, myosin phosphatase Rho-interacting protein, collagen alpha-6(IV) chain protein FAM110A, DNA polymerase epsilon catalytic subunit A, semaphorin-5B, ephrin type-B receptor 1, zinc finger protein 532, tyrosine-tRNA ligase (cytoplasmic), mismatch repair endonuclease PMS2, cadherin EGF LAG seven-pass G-type receptor, cytoskeleton-associated protein 2, tyrosine-tRNA ligase (cytoplasmic), zinc finger protein 618, cytoskeleton-associated protein 2, alpha-2-macroglobulin, serine/threonine-protein kinase OSR1, collagen alpha-1(XII) chain, zinc finger ZZ-type and EF-hand domain-containing protein 1, iporin, phospholipid-transporting ATPase IG, collagen alpha-6(IV) chain, spatacsin, short stature homeobox protein 2, collagen alpha-1(XII) chain, ubiquitin carboxyl-terminal hydrolase 28, phospholipid-transporting ATPase IG, cyclin-dependent kinase 13, A-kinase anchor protein 9, zinc finger protein 417, sorting nexin-13, hemoglobin subunit beta, hemoglobin subunit delta, hemoglobin subunit alpha, cytochrome P450 3A4 isoform, filamin-C, apolipoprotein A-IV, nebulin, SAA2-SAA2 protein precursor, plasminogen isoform 1 precursor, ATPase family AAA domain-containing protein 5, DNA damage-induced apoptosis suppressor protein, E3 ubiquitin-protein ligase Midline-1, mitogen-activated protein kinase kinase kinase kinase 1, mucin-16, probable E3 ubiquitin-protein ligase HECTD4, protein RRPS homolog, retrotransposon Gag-like protein 9, serine/threonine-protein phosphatase 2A regulatory subunit B″ subunit gamma, and uncharacterized protein C12orf42.

In one aspect, the biomarker may be selected from one or more of Ephrin B2, Epidermal Growth Factor, Ferritin-heavy chain, Apotransferrin, Phosphoinositide 3-kinase, Mdm2-binding protein isoform X3, Tubulin polyglutamylase TTLL13 isoform X7, Heparin sulfate glucosamine 3-O-sulfotransferase 2.

In one aspect, the characterization of the disclosed method may be identifying an individual as having improved disease, progressing disease, or a plateau in disease.

In one aspect, an alteration or change in a biomarker level compared to a control level indicates one or both of the presence of disease or progression of disease.

In one aspect, the method may comprise a second comparison, wherein the second comparison is carried out following administration of a treatment. In this aspect, a return, or a trend to a return, to a biomarker level to that of a control value indicates an improvement in disease. In other words, following administration of a treatment, a second comparison in which an aberrant or abnormal biomarker returns to a normal level (a normalization of the biomarker) indicates that the treatment is effective. In such cases, the treatment may be continued until complete normalization. In further aspects, if a normalization or trend towards normalization does not occur, then an increased dose or frequency of the treatment may be administered, or alternatively, an alternative treatment may be used.

In one aspect, the pulmonary fibrosis disease may be selected from progressive pulmonary fibrosis, interstitial lung disease (ILD), and idiopathic pulmonary fibrosis (IPF).

In one aspect, the treatment may be any treatment known to be efficacious for a pulmonary fibrosis disease. In certain aspects, for example, the treatment may be selected from one or more of a PI3K pathway inhibitor, an EGF pathway inhibitor, pirfenidone (a medication used for the treatment of idiopathic pulmonary fibrosis that works by reducing lung fibrosis through downregulation of the production of growth factors and procollagens I and II), nintendanib (sold under the brand names Ofev and Vargatef, an oral medication used for the treatment of idiopathic pulmonary fibrosis and for some types of non-small-cell lung cancer), and combinations thereof). Exemplary EGF pathway inhibitors include erlotinib, cetuximab, panitumumab, gefitinib, erlotinib, lapatinib, canertinib, and combinations thereof. Exemplary P13K pathway inhibitors include alpelisib, taselisib, idelalisib, and compbinations thereof.

In one aspect, the individual may be one having, or at high-risk of having or of developing idiopathic pulmonary fibrosis (IPF), wherein the methods are used to determine disease status. Disease status may include having a trajectory of improvement, disease progression, or disease stagnation, particularly with respect to IPF status. In one aspect, the high-risk individual is one having one or more predispositions selected from a familial history of IPF, age of 60 years or greater, and a history of chronic smoking.

In one aspect, a method for monitoring idiopathic pulmonary fibrosis (IPF) progression in an individual in need thereof is disclosed. In this aspect, the method may comprise assaying one or more of the aforementioned biomarkers.

In one aspect, a method for testing a potential therapeutic in an individual having idiopathic pulmonary fibrosis (IPF) is disclosed, in which the method may comprise assaying one or more biomarkers at a time point selected from one or more of before, during, or after administration of a potential therapeutic agent, wherein a normalization of a level of a biomarker following treatment indicates that the potential therapeutic has efficacy in treating IPF. The methods may be further used to assist in dosage, including amount and frequency of the dose, wherein the methods may be used to determine whether an increased or more frequent dosage contributes to an improved or enhanced normalization of an abnormal biomarker level. Yet further, using the methods, it may be determined that a lesser dosage or frequency may be used to obtain a similarly efficacious result of normalizing biomarkers (and in turn, disease status or progression).

In one aspect, a method of diagnosing and treating a pulmonary disease as disclosed herein, for example, idiopathic pulmonary fibrosis (IPF) in an individual in need thereof is disclosed. In this aspect, the method may comprise
a) establishing, using a computing system, a model for characterizing the disease state of an individual having or likely to have a pulmonary disease, by using expression level data of one or more of the disclosed biomarkers;
b) contacting a biological sample from said individual with at least one detection agent capable of specifically binding to said one or more biomarkers;
c) acquiring an expression level from said one or more biomarkers measured from said biological sample;
d) characterizing said individual as one or more of “likely to have a lung disease,” “having stable lung disease,” “having progressive lung disease,” or “having improving lung disease;” and
e) optionally, administering a therapy to said individual based on said characterization.

### Detection Methods

The disclosed methods may use any methods known in the art for detection of a biomarker. The methods may include the diagnosis, prognosis, stratification and/or monitoring of disease in a human subject, and may, in certain aspects comprise a) detecting a level of one or more biomarker proteins in a biological sample obtained from the human subject, wherein a higher or lower level of the one or more biomarker protein in the biological sample from the human subject compared to a reference value (such as that of a control subject that does not have the disease state of interest) is indicative of the state of the disease, for example, a pulmonary fibrosis disorder, more particularly, in certain aspects, IPF. The state of a pulmonary disorder that may be determined using the methods may include the propensity or likelihood of developing the disorder, the progression of the disorder, or the resolution of the disorder, with or without a therapeutic intervention.

The disclosed methods may employ a variety of different methods for detecting one or more biomarkers and/or quantifying or determining a relative level of one or more biomarkers. The methods may include protein level measurements, DNA measurements, or RNA measurements. In certain exemplary aspects, the one or more biomarkers may be detected as proteins, which may be detected using one or more antibodies specific for the biomarker protein as is described herein, for example, one or more of western blot, ELISA, Proximity Extension Assay, or mass-spectrometrically, though it is expressly noted that any method sufficient to distinguish levels of the aforementioned one or more biomarkers, whether as protein or mRNA, or any other measure of gene expression, may be used with the disclosed methods and the examples herein are not intended to limit the scope of the invention.

Methods for capturing, analyzing, quantifying, etc., biomarkers are known in the art and may be used in conjunction with the disclosed methods. For example, in one aspect, one or more of the disclosed biomarkers may be captured with capture reagents immobilized to a solid support, such as a biochip or other substrate suitable for such capture, and may include, for example, a glass or plastic in the form of, e.g., a microtiter plate, a stick, a bead, or a microbead. Once captured on a substrate, the marker or markers in a sample may be assayed for presence and/or quantity/abundance. In one aspect, one or more of the disclosed biomarkers may be detected and/or measured by a variety of detection methods including for example, gas phase ion spectrometry methods, optical methods, electrochemical methods, atomic force microscopy and radio frequency methods.

In one aspect, a sample obtained from an individual may be prepared to enhance detectability of one or more biomarkers therein. For example, a blood serum sample may be fractionated using methods known in the art. Examples in include Cibacron blue agarose chromatography and single stranded DNA affinity chromatography, anion exchange chromatography, affinity chromatography (e.g., with antibodies) and the like. Such fractionation may be carried out prior to detection of the biomarker. The method of fractionation may depend on the type of detection method used. Methods that enriches for the protein of interest can be used. Sample preparations, such as pre-fractionation protocols, are optional and may not be necessary to enhance detectability of markers depending on the methods of detection used. In one aspect, biomarkers in a sample may be separated by high-resolution electrophoresis, e.g., one or two-dimensional gel electrophoresis. A fraction containing a marker can be isolated and further analyzed by gas phase ion spectrometry. In another embodiment, two-dimensional gel electrophoresis is used to generate two-dimensional array of spots of biomarkers, including one or more markers.

In one aspect, an immunoassay may be used to detect and analyze a biomarker in a sample. In this aspect, the method may comprise (a) providing an antibody that specifically binds to a marker; (b) contacting a sample with the antibody; and (c) detecting the presence of a complex of the antibody bound to the marker in the sample. For example, an exemplary immunoassay employs an antibody to specifically bind an antigen (e.g., a marker), and uses the specific binding properties of a particular antibody to isolate, target, and/or quantify the antigen. The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. For example, polyclonal antibodies can be selected to obtain only those polyclonal antibodies that are specifically immunoreactive with that marker and not with other proteins, except for polymorphic variants and alleles of the marker. This selection may be achieved by subtracting out antibodies that cross-react with the marker molecules from other species.

In one aspect, the method of detection may employ a labeled detection reagent. For example, the detection reagent may be, e.g., a first or second antibody labeled with a detectable label. Exemplary detectable labels include magnetic beads (e.g., DYNABEADS™), fluorescent dyes, radiolabels, enzymes (e.g., horse radish peroxide, alkaline phosphatase and others commonly used in an ELISA), and colorimetric labels such as colloidal gold or colored glass or plastic beads. Alternatively, the marker in the sample can be detected using an indirect assay, wherein, for example, a second, labeled antibody may used to detect bound marker-specific antibody, and/or in a competition or inhibition assay wherein, for example, a monoclonal antibody which binds to a distinct epitope of the marker may incubated simultaneously with the mixture.

Exemplary methods for measuring the amount of, or presence of, one or more biomarkers include detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Electrochemical methods include voltametry and amperometry methods. Radio frequency methods include multipolar resonance spectroscopy. Methods for performing these assays are known in the art and may include an enzyme immune assay (EIA) such as enzyme-linked immunosorbent assay (ELISA), a radioimmune assay (RIA), a Western blot assay, or a slot blot assay.

In one aspect, the method may employ an incubation step or a washing step. For example, incubation steps may vary from about 5 seconds to several hours, or from about 5 minutes to about 24 hours. The incubation time will depend upon the assay format, marker, volume of solution, concentrations, and the like. The methods may be carried out at ambient temperature, or may be conducted over a range of temperatures, such as 10° C. to 40° C.

In one aspect, the methods employ an immunoassays to determine presence or absence of a marker in a sample, as well as the quantity of a marker in a sample, in which the amount of an antibody-marker complex may be determined by comparing to a standard. A standard can be, e.g., a known compound or another protein known to be present in a sample which would be expected to remain unchanged in the presence of the disease state being evaluated. The test amount of marker need not be measured in absolute units, as long as the unit of measurement can be compared to a control.

In one aspect, the biomarker may be detected, and biomarker data may be generated, wherein the data may then be analyzed by a computer software program. The software may comprise code that converts signal from the mass spectrometer into computer readable form, and may include code that applies an algorithm to the analysis of the signal to determine whether the signal represents a “peak” in the signal corresponding to one or more markers as disclosed herein. The software may also include code that executes an algorithm that compares signal from a test sample to a typical signal characteristic of a “normal” sample obtained from an individual known to not have a disease state contemplated herein, and determines the closeness of fit between the two signals. The software also can include code indicating which the test sample is closest to, thereby providing a probable diagnosis, trajectory, and/or disease outcome.

### Kits

In one embodiment, provided herein are kits comprising reagents for detecting one or more biomarker levels, wherein the reagents may include antibodies and/or nucleic acids, which may hybridize to the mRNA or protein of the biomarker(s) of a biological sample. Reagents may be labelled for subsequent detection, wherein the detection allows for measurement, whether absolute or relative, of the biomarker level. In other aspect, the kit may include instructions for detecting the label qualitatively or quantitatively. In another aspects, disclosed are kits for using the disclosed biomarkers which further include assays and analytical tools for the assays, such as one or more of reagents, standards and instructions for analyzing the expression level of one or more biomarkers in a biological sample. The kit may comprise, in certain aspects, a buffering agent, a preservative, or a protein stabilizing agent, an enzyme, or a substrate. The substrate may be a means of detecting a label, or the expressed biomarker protein product itself, which may further include mRNA associated with the biomarker protein. In one aspect, the kit may comprise reagents that may be necessary for detection of nucleic acids, amino acids or hybridization signals for nucleic acids.

In one aspect, the results obtained may be compared to a standard, which, may, for example, comprise a series of standards. The standard(s) may be used in the kits for quantification of differential levels of the biomarker or differential expression. In one aspect, the standard may comprise antibodies for detecting a standard biomarker. In one aspect, the standard may comprise nucleic acids when the kit is used for the determination of nucleic acid profile, or in another aspect the standard is a protein when the kit is used for the determination of expressed protein profile. The kit may be adapted for high-throughput screening, and comprise a microarray. In certain aspects, the kit may comprise a microarray, which may comprise cRNA of the genes indicated, and others. In one aspect, the kit may comprise one or more of standard oligonucleotide probes, PCR reagents and detectable labels. The kit further may comprise a positive and negative control, wherein said standards can be assayed and compared to the test sample. Kits that include one or more reaction vessels that have aliquots of some or all of the reaction components of the invention in them may be provided. Aliquots can be in liquid or dried form. Reaction vessels can include sample processing cartridges or other vessels that allow for the containment, processing and/or amplification of samples in the same vessel.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

The instant disclosure relates to methods for the characterization and/or treatment of pulmonary disease and fibrosis using biomarkers of pulmonary injury or early predictors of interstitial lung disease. Further disclosed are improved tools to study pulmonary fibrosis and evaluate therapeutics.

To guide in the selection of candidate plasma biomarkers of pulmonary fibrosis, Applicant studied a transgenic mouse model of progressive pulmonary fibrosis where fibrosis is induced by overexpressing transforming growth factor-alpha (TGFα). Mice in this model develop histological, bio-chemical and physiological changes similar to those seen in human fibrotic disease including gene expression profiles, migrating fibrotic lesions, severe restrictive changes in lung mechanics, cachexia and ultimately death from a progressive fibrotic burden.⁸ Despite the severe and progressive nature of the fibrosis, these lesions are partially reversible when the transgene or downstream signaling pathways are extinguished.⁹ This unique feature of the model allows for identification of biomarkers of fibrosis progression and resolution.

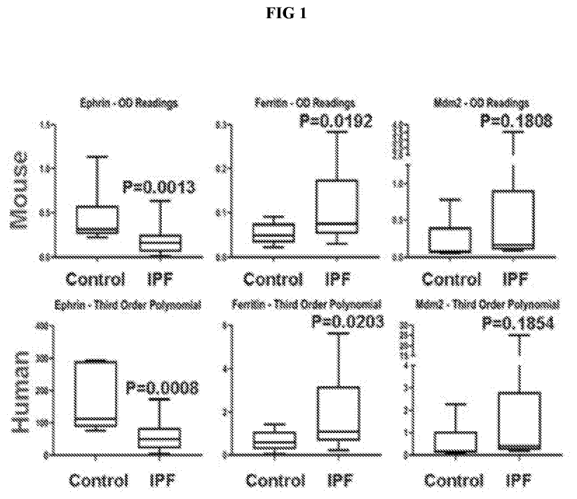

In preliminary data, Applicant performed mass spectrometry on mouse plasma from TGFα mice and identified candidate proteins that were altered during the progression of fibrosis and returned to baseline during regression. Through our collaboration with the University of Michigan we next obtained plasma from a longitudinal cohort of carefully phenotyped patients with IPF, The COMET Study. The COMET study was a multi-center, observational cohort of highly characterized IPF patients followed prospectively up to 80 weeks. All subjects underwent baseline assessment, including demographics, patient-reported descriptors, PFTs and plasma samples which were prospectively repeated at 16-week intervals. Within this cohort are patients whose disease progressed during the 80-week period and patients who were stable. These samples thus represented a highly unique and valuable resource for identifying plasma protein changes associated with physiologic and clinical changes in IPF disease progression. To determine potential relevance of murine plasma candidates with human disease, Applicant identified Ephrin-B2, Mdm2-binding protein and ferritin heavy chain, all proteins which have been shown to mediate fibrogenesis in IPF cell lines or experimental models of fibrosis in the liver and kidney¹⁰⁻¹¹. Applicant then performed ELISA for Ephrin, Mdm2 and ferritin on IPF plasma samples and normal human controls and compared with TGFα mice. ELISA results in the IPF group were altered from controls mirroring differences in the fibrotic mouse model (FIG. 1).

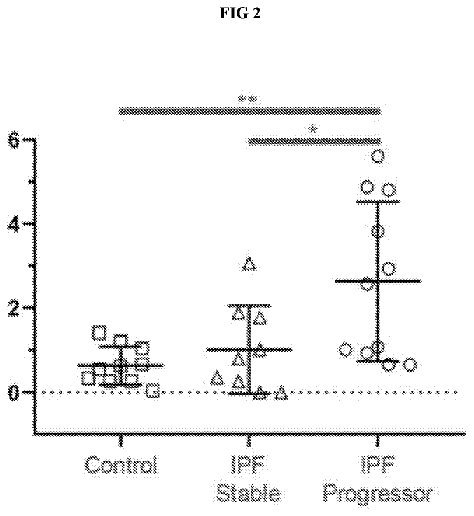

Among the samples analyzed by ELISA were subgroups of patients whose fibrotic disease was stable or progressed. When comparing ferritin levels among these subgroups, those individuals whose disease advanced had higher levels compared with patients whose disease remained stable (FIG. 2). Taken together, this preliminary data demonstrates a strong correlation in altered plasma proteins identified by mass spectrometry in the TGFα mouse model with HT plasma samples. These findings demonstrate the feasibility that a murine model combined with human samples can identify specific plasma markers of pulmonary fibrosis progression.

Applicant then performed an unbiased mass spectrometry analysis on the plasma of 35 COMET Study patients and 17 age-matched controls. Using the data from this proteomic analysis, Applicant identified additional candidate proteins associated with pulmonary fibrosis (Table 1). Pathway analysis of these biomarkers revealed dysfunction in a number of pathways that can be modulated with FDA approved drug therapeutics (Table 2).

Exemplary inhibitors of the PI3K pathway include alpelisib, taselisib, and idelalisib. Alpelisib (BYL719; Novartis Pharmaceuticals, Basel, Switzerland) is the first oral PI3Ki to selectively target the class I p110α-isoform (IC50=4.6 nM). A phase I trial (NCT01219699) included patients with PIK3CA-altered advanced solid tumors and showed sensitivity to alpelisib monotherapy. See, e.g. LoRusso P M. Inhibition of the PI3K/AKT/mTOR Pathway in Solid Tumors. J Clin Oncol. 2016 Nov 1; 34(31):3803-3815. doi: 10.1200/JCO.2014.59.0018. Epub 2016 Sep 30. PMID: 27621407; PMCID: PMC6366304. Taselisib (GDC-0032, Genentech, San Francisco, CA) is an oral class I PI3Ki, sometimes referred as β-sparing, as it exhibits equipotent inhibition of p110α, p110-γ and p110-δ, but inhibits p110β with 30-fold lower potency. Idelalisib (Zydelig tablets; Gilead Sciences, Inc.) described chemically as 5-fluoro-3-phenyl-2-[(1S)-1-(9H-purin-6-yl-amino)propyl] quinazolin-4(3H)-one, is a lipid kinase inhibitor of the class I phophatidylinositol-3 kinase p110-δ (PI3K-δ). See, e.g., Clin Cancer Res Apr. 1, 2015 (21) (7) 1525-1529; DOI: 10.1158/1078-0432.CCR-14-2522

Exemplary inhibitors of the EGF pathway include erlotinib, cetuximab, panitumumab, gefitinib, erlotinib, lapatinib, and canertinib. See, for example, Seshacharyulu P, Ponnusamy M P, Haridas D, Jain M, Ganti A K, Batra S K. Targeting the EGFR signaling pathway in cancer therapy. Expert Opin Ther Targets. 2012 Jan.; 16(1):15-31. doi: 10.1517/14728222.2011.648617. Epub 2012 Jan. 12. PMID: 22239438; PMCID: PMC3291787, describing such agents. Gifitinib (ZD1839/Iressa): Gefitinib is an anilinoquinazoline derived EGFR tryrosine kinase inhibitor and was first characterized in the year 1996. It is an orally active low-molecular-weight EGFR inhibitor with selective tyrosine kinase activity but does not inhibit serine-threonine kinase activity. Gefitinib has a 200-fold greater affinity for EGFR relative to the other ErbB family members. Erlotinib (also referred to as OSI-774, or Tarceva, Erlotinib hydrochloride is another FDA-approved low molecular weight molecule similar to gefitinib, available in the form of an orally potent and selectively reversible inhibitor of EGFR tyrosine kinase). Gefitinib (ZD1839/Iressa): Gefitinib is an anilinoquinazoline derived EGFR tryrosine kinase inhibitor and was first characterized in the year 1996. It is an orally active low-molecular-weight EGFR inhibitor with selective tyrosine kinase activity but does not inhibit serine-threonine kinase activity. Lapatinib (GW-572016): Lapatinib is an orally active, reversible and specific RTK inhibitor of both EGFR and HER2 as well it was also found to exhibit activity against an found to have activity against AKT overexpressing human tumor xenografts. Due to its nonselective nature of EGFR inhibition, it accounts for a broader spectrum of anti-tumor activity with improved efficacy. Canertinib (CI-1033): Canertinib is a 3-chloro 4-fluoro 4-anilinoquinazoline compound. It is an orally active low-molecular-weight irreversible pan-EGFR family TKI.

TABLE 1
Plasma proteins changed in human IPF samples
and/or mouse TGF-alpha fibrosis model

| Marker | Function | Association with Fibrosis |
|---|---|---|
| Ephrin B2 | Cell surface transmembrane | Pro-fibrotic role of Ephrin |
| | ligand for Ephrin receptors. | B2 in cardiac and liver |
| | Plays key role in cell | |
| | migration | |
| Epidermal Growth Factor | Ligand that binds EGFR. | Significantly differs in IPF |
| | Stimulates the growth of | |
| | various epidermal and | |
| | epithelial tissues | |
| Ferritin-heavy chain | Stores iron in a soluble, non- | Elevated serum ferritin is an |
| | toxic, readily available form | independent predictor of |
| | | histologic severity and |
| | | advanced liver fibrosis |
| Apotransferrin | Binds iron. Facilitates iron | Marker of liver fibrosis |
| | transport | |
| Phosphoinositide 3-kinase | Regulates cell proliferation | Involved in MTOR |
| | and migration | regulation in fibrosis |
| Mdm2-binding protein | Inhibits cell migration | Significantly Changed in |
| isoform X3 | | epithelial cells from patients |
| | | with IPF |
| Tubulin polyglutamylase | Modifies alpha-tubulin. | Involved in lung remodeling |
| TTLL13 isoform X7 | Involved in elongation of the | |
| | polyglutamylation reaction | |
| Heparin sulfate glucosamine | Utilizes 3′-phospho- | Significantly changed in |
| 3-O-sulfotransferase 2 | 5′adenylyl sulfate (PAPS) to | kidney fibrosis |
| | catalyze the transfer of a | |
| | sulfo group | |

TABLE 2
Targetable pathways enriched by markers.

| Pathway | FDR-corrected P value |
|---|---|
| P13K signaling and response to cAMP | 3.918e−14 |
| Iron transport and homeostasis | 1.841e−13 |
| Platelet activation | 2.039e−11 |
| Blood coagulation | 1.368e−10 |
| Cellular adhesion and integrin priming | 4.168e−10 |
| Cellular proliferation and apoptosis | 4.0583−7 |
| Interleukin 10 anti-inflammatory signaling | 1.853e−5 |

TABLE 3
Additional IPF Biomarkers. The category of “IPF v Non IPF”
are those proteins present in IPF and controls but more different
in IPF than non-IPF controls. “Present only in IPF”
is proteins only seen in IPF and not in controls while “Missing
in IPF” is the corollary.

idiopathic pulmonary fibrosis (IPF) v Non idiopathic pulmonary
fibrosis (IPF)
pregnancy zone protein
serotransferrin
alpha-2-macroglobulin
immunoglobulin lambda-like polypeptide 5
ceruloplasmin
alpha-1-antitrypsin precursor
complement C3 preproprotein
coiled-coil domain-containing protein 144A
cytochrome P450 3A43
myosin phosphatase Rho-interacting protein
collagen alpha-6(IV) chain
protein FAM110A
DNA polymerase epsilon catalytic subunit A
semaphorin-5B
ephrin type-B receptor 1
zinc finger protein 532
tyrosine--tRNA ligase, cytoplasmic
mismatch repair endonuclease PMS2
cadherin EGF LAG seven-pass G-type receptor
cytoskeleton-associated protein 2
tyrosine-tRNA ligase, cytoplasmic
zinc finger protein 618
cytoskeleton-associated protein 2
alpha-2-macroglobulin
serine/threonine-protein kinase OSR1
collagen alpha-1(XII) chain
zinc finger ZZ-type and EF-hand domain-containing protein 1
iporin
phospholipid-transporting ATPase IG
collagen alpha-6(IV) chain
Spatacsin
short stature homeobox protein 2
collagen alpha-1(XII) chain
ubiquitin carboxyl-terminal hydrolase 28
phospholipid-transporting ATPase IG
cyclin-dependent kinase 13
A-kinase anchor protein 9
zinc finger protein 417
sorting nexin-13
Present only in idiopathic pulmonary fibrosis (IPF):
hemoglobin subunit beta
hemoglobin subunit delta
hemoglobin subunit alpha
cytochrome P450 3A4 isoform
filamin-C
apolipoprotein A-IV
nebulin
SAA2-SAA2 protein precursor
plasminogen isoform 1 precursor
Missing in idiopathic pulmonary fibrosis (IPF):
ATPase family AAA domain-containing protein 5
DNA damage-induced apoptosis suppressor protein
E3 ubiquitin-protein ligase Midline-1
mitogen-activated protein kinase kinase kinase kinase 1
mucin-16
probable E3 ubiquitin-protein ligase HECTD4
protein RRP5 homolog
retrotransposon Gag-like protein 9
serine/threonine-protein phosphatase 2A regulatory subunit
B″ subunit gamma
ubiquitin carboxyl-terminal hydrolase 28
uncharacterized protein C12orf42

The underlying hypothesis of Applicant's efforts is there are specific proteins which are altered in the plasma of patients with progressive pulmonary fibrotic disease which can be detected by mass spectrometry. A unique plasma biomarker profile may be useful to clinicians and clinical researchers to help diagnosis fibrotic disease, monitor disease progression and investigate the efficacy of antifibrotic therapies. The concept of measuring plasma protein biomarker panels to detect or monitor disease is already in practice in multiple processes with high accuracy¹³. However, there are currently no commercial tests blood tests which can monitor pulmonary fibrosis.

Combining Applicant's previous identification of plasma proteins from the murine TGF model with larger-scale mass spectrometry analysis of IPF and control samples, Applicant has now identified candidate proteins which may be used to discriminate pulmonary fibrosis from healthy lung and/or to evaluate progression of disease. Progression of disease can be defined as any of the following: death, acute exacerbation of IPF, lung transplant, or relative change in FVC of 10% or DLCO of 15%. Temporal association of blood draws and cliaanical assessment allows comparison of the timing of changes in plasma proteins with disease course. In sum, the identified markers of pulmonary fibrosis disease progression may be obtained rapidly, noninvasively and directly reflect the underlying pathophysiology of the disease process.

The identified biomarkers may be used for one or more of the following:

Monitoring disease (e.g., IPF) progression: Pulmonary fibrotic diseases including IPF will often intermittently progress. A sensitive plasma profile may provide improved guidance for clinical management decisions. For instance, a plasma profile indicating alterations in fibrotic proteins would indicate early progression of disease which may not be detected immediately by PFT or HRCT. Such findings may then be use for more rapid changes in initiation or adjustment of medications or earlier referral for lung transplantation. Alternatively, if a plasma profile is unaltered, these findings would suggest disease stability and no changes in management.

Early disease (e.g., IPF) diagnosis: A plasma profile specific for the development or progression of fibrotic disease would be highly valuable for early discrimination of pulmonary fibrosis in various disease states such as interstitial lung diseases (ILD). There are over 200 ILD, many of which have the potential to develop fibrosis as a complicating feature. A blood proteome signature specific for fibrosis would be an important diagnostic adjunct by allowing the clinician to determine if pulmonary fibrosis is a component of the ILD. Results from this assay would be useful in assisting with the determination if pulmonary fibrosis is present or developing and guiding in the management of antifibrotic therapy.

Companion diagnostic: Plasma biomarkers of fibrosis progression would provide a valuable tool for clinical researchers. Progression of fibrosis is often an insidious process developing over several years and current markers of progression are indirect and insensitive. Consequently, clinical trials often need to have large sample sizes and be of long duration adding significant cost and potentially reducing the number of promising agents which could be entered into early clinical trials. This decade will likely introduce several novel and promising antifibrotic compounds for the treatment of pulmonary fibrosis. Providing clinical researchers with sensitive plasma markers correlated with fibrosis progression will potentially enable more novel compounds to enter clinical trials by reducing the number of patients, length of time and subsequent cost currently needed to assess efficacy.

### REFERENCES

1. Hardie W D, Glasser S W, Hagood J S. Emerging concepts in the pathogenesis of lung fibrosis. 2009;175(1):3-16.

2. Miller M R, Hankinson J, Brusasco V, et al. Standardisation of spirometry. 2005;26(2):319-338.

3. Pellegrino R, Viegi G, Brusasco V, et al. Interpretative strategies for lung function tests. 2005;26(5):948-968.

4. Lynch D A, Godwin J D, Safrin S, et al. High-resolution computed tomography in idiopathic pulmonary fibrosis: diagnosis and prognosis. 2005;172(4):488-493.

5. Wu X, Kim G H, Salisbury M L, et al. Computed Tomographic Biomarkers in Idiopathic Pulmonary Fibrosis. The Future of Quantitative Analysis. 2019;199(1):12-21.

6. Cottin V. Lung biopsy in interstitial lung disease: balancing the risk of surgery and diagnostic uncertainty. 2016;48(5):1274-1277.

7. Shah R, Patel T, Freedman J E. Circulating Extracellular Vesicles in Human Disease. 2018;379(10):958-966.

8. Hardie W D, Korfhagen T R, Sartor M A, et al. Genomic profile of matrix and vasculature remodeling in TGF-alpha induced pulmonary fibrosis. 2007;37(3):309-321.

9. Hardie W D, Kerlakian C B, Bruno M D, et al. Reversal of lung lesions in transgenic transforming growth factor alpha mice by expression of mutant epidermal growth factor receptor. 1996;15(4):499-508.

10. Nakashima N, Kuwano K, Maeyama T, et al. The p53-Mdm2 association in epithelial cells in idiopathic pulmonary fibrosis and non-specific interstitial pneumonia. 2005;58(6) :583-589.

11. Lagares D, Ghassemi-Kakroodi P, Tremblay C, et al. ADAM10-mediated ephrin-B2 shedding promotes myofibroblast activation and organ fibrosis. 2017;23(12):1405-1415.

12. Kowdley K V, Belt P, Wilson L A, et al. Serum ferritin is an independent predictor of histologic severity and advanced fibrosis in patients with nonalcoholic fatty liver disease. 2012;55(1):77-85.

13. Jung Y J, Katilius E, Ostroff R M, et al. Development of a Protein Biomarker Panel to Detect Non-Small-Cell Lung Cancer in Korea. 2017;18(2):e99-e107.

14. Madala S K, Thomas G, Edukulla R, et al. p70 ribosomal S6 kinase regulates subpleural fibrosis following transforming growth factor-alpha expression in the lung. 2016;310(2): L175-186.

15. Madala S K, Schmidt S, Davidson C, Ikegami M, Wert S, Hardie W D. MEK-ERK pathway modulation ameliorates pulmonary fibrosis associated with epidermal growth factor receptor activation. 2012;46(3):380-388.

16. Madala S K, Korfhagen T R, Schmidt S, et al. Inhibition of the alphavbeta6 integrin leads to limited alteration of TGF-alpha-induced pulmonary fibrosis. 2014;306(8):L726-735.

17. Madala S K, Edukulla R, Phatak M, et al. Dual targeting of MEK and PI3K pathways attenuates established and progressive pulmonary fibrosis. 2014;9(1):e86536.

18. Le Cras T D, Korfhagen T R, Davidson C, et al. Inhibition of PI3K by PX-866 prevents transforming growth factor-alpha-induced pulmonary fibrosis. 2010;176(2):679-686.

19. Korfhagen T R, Le Cras T D, Davidson C R, et al. Rapamycin prevents transforming growth factor-alpha induced pulmonary fibrosis. 2009;41(5):562-572.

20. Hardie W D, Davidson C, Ikegami M, et al. EGF receptor tyrosine kinase inhibitors diminish transforming growth factor-alpha-induced pulmonary fibrosis. 2008;294(6):L1217-1225.

21. Krefft S D, Rose C S, Nawaz S, Miller Y E. Deployment-Related Lung Disorders. 2015;32(6):32-38.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method for assessing pulmonary fibrosis disorder disease status in an individual in need thereof, comprising
a. detecting a level of one or more biomarkers selected from Ephrin B2, Epidermal Growth Factor, Ferritin-heavy chain, Apotransferrin, Phosphoinositide 3-kinase, Mdm2-binding protein isoform X3, Tubulin polyglutamylase TTLL13 isoform X7, Heparin sulfate glucosamine 3-O-sulfotransferase 2, in a biological sample obtained from said individual;
b. comparing said level to that of a control value;
c. characterizing the disease status in said individual; and
d. administering a treatment to said individual, wherein said treatment is determined based on the characterization of step c.

2. The method of claim 1, further comprising detecting a biomarker selected from pregnancy zone protein, serotransferrin, alpha-2-macroglobulin, immunoglobulin lambda-like polypeptide 5, ceruloplasmin, alpha-1-antitrypsin precursor, complement C3 preproprotein, coiled-coil domain-containing protein 144A, cytochrome P450 3A43, myosin phosphatase Rho-interacting protein, collagen alpha-6(IV) chain protein FAM110A, DNA polymerase epsilon catalytic subunit A, semaphorin-5B ephrin type-B receptor 1, zinc finger protein 532, tyrosine-tRNA ligase (cytoplasmic), mismatch repair endonuclease PMS2, cadherin EGF LAG seven-pass G-type receptor, cytoskeleton-associated protein 2, tyrosine-tRNA ligase (cytoplasmic), zinc finger protein 618, cytoskeleton-associated protein 2, alpha-2-macroglobulin, serine/threonine-protein kinase OSR1, collagen alpha-1(XII) chain, zinc finger ZZ-type and EF-hand domain-containing protein 1, iporin, phospholipid-transporting ATPase IG, collagen alpha-6(IV) chain, spatacsin, short stature homeobox protein 2, collagen alpha-1(XII) chain, ubiquitin carboxyl-terminal hydrolase 28, phospholipid-transporting ATPase IG, cyclin-dependent kinase 13, A-kinase anchor protein 9, zinc finger protein 417, sorting nexin-13, hemoglobin subunit beta, hemoglobin subunit delta, hemoglobin subunit alpha, cytochrome P450 3A4 isoform, filamin-C, apolipoprotein A-IV, nebulin, SAA2-SAA2 protein precursor, plasminogen isoform 1 precursor, ATPase family AAA domain-containing protein 5, DNA damage-induced apoptosis suppressor protein, E3 ubiquitin-protein ligase Midline-1, mitogen-activated protein kinase kinase kinase kinase 1, mucin-16, probable E3 ubiquitin-protein ligase HECTD4, protein RRP5 homolog , retrotransposon Gag-like protein 9, serine/threonine-protein phosphatase 2A regulatory subunit B″ subunit gamma, and uncharacterized protein C12orf42.

3. The method of claim 1, wherein said characterization comprises identifying said individual as having improved disease, progressing disease, or a plateau in disease.

4. The method of claim 1, wherein an alteration in biomarker level compared to said control level indicates the presence of disease or progression of disease.

5. The method of claim 1, wherein a second comparison step is carried out following administration of said treatment, wherein a return to said biomarker level to that of a control value indicates an improvement in disease.

6. The method of claim 1, wherein said pulmonary fibrosis disease is selected from progressive pulmonary fibrosis, interstitial lung disease (ILD), and idiopathic pulmonary fibrosis (IPF).

7. The method of claim 1, wherein said treatment is selected from a PI3K pathway inhibitor, an EGF pathway inhibitor, Pirfenidone, Nintendanib, and combinations thereof, preferably wherein said EGF pathway inhibitor is selected from erlotinib, cetuximab, panitumumab, gefitinib, erlotinib, lapatinib, and canertinib, preferably wherein said P13K pathway inhibitor is selected from alpelisib, taselisib, and idelalisib.

8. The method of claim 1, wherein said individual has, or is at high-risk or of developing idiopathic pulmonary fibrosis (IPF), and wherein said disease status is selected from having a trajectory of improvement, progression, or stagnation of IPF disease.

9. The method of claim 8 wherein said individual has one or more predispositions selected from a familial history of IPF, age of 60 years or greater, and a history of chronic smoking.

10. A method for monitoring idiopathic pulmonary fibrosis (IPF) progression in an individual in need thereof comprising assaying one or more biomarkers selected from Ephrin B2, Epidermal Growth Factor, Ferritin-heavy chain, Apotransferrin, Phosphoinositide 3-kinase, Mdm2-binding protein isoform X3, Tubulin polyglutamylase TTLL13 isoform X7, Heparin sulfate glucosamine 3-O-sulfotransferase 2, serotransferrin, alpha-2-macroglobulin, immunoglobulin lambda-like polypeptide 5, ceruloplasmin, alpha-1-antitrypsin precursor, complement C3 preproprotein, coiled-coil domain-containing protein 144A, cytochrome P450 3A43, myosin phosphatase Rho-interacting protein, collagen alpha-6(IV) chain protein FAM110A, DNA polymerase epsilon catalytic subunit A, semaphorin-5B, ephrin type-B receptor 1, zinc finger protein 532, tyrosine-tRNA ligase (cytoplasmic), mismatch repair endonuclease PMS2, cadherin EGF LAG seven-pass G-type receptor, cytoskeleton-associated protein 2, tyrosine-tRNA ligase (cytoplasmic), zinc finger protein 618, cytoskeleton-associated protein 2, alpha-2-macroglobulin, serine/threonine-protein kinase OSR1, collagen alpha-1(XII) chain, zinc finger ZZ-type and EF-hand domain-containing protein 1, iporin, phospholipid-transporting ATPase IG, collagen alpha-6(IV) chain, spatacsin, short stature homeobox protein 2, collagen alpha-1(XII) chain, ubiquitin carboxyl-terminal hydrolase 28, phospholipid-transporting ATPase IG, cyclin-dependent kinase 13, A-kinase anchor protein 9, zinc finger protein 417, sorting nexin-13, hemoglobin subunit beta, hemoglobin subunit delta, hemoglobin subunit alpha, cytochrome P450 3A4 isoform, filamin-C, apolipoprotein A-IV, nebulin, SAA2-SAA2 protein precursor, plasminogen isoform 1 precursor, ATPase family AAA domain-containing protein 5, DNA damage-induced apoptosis suppressor protein, E3 ubiquitin-protein ligase Midline-1, mitogen-activated protein kinase kinase kinase kinase 1, mucin-16, probable E3 ubiquitin-protein ligase HECTD4, protein RRP5 homolog , retrotransposon Gag-like protein 9, serine/threonine-protein phosphatase 2A regulatory subunit B″ subunit gamma, and uncharacterized protein C12orf42.

11. A method for testing a potential therapeutic in an individual having idiopathic pulmonary fibrosis (IPF) comprising assaying one or more biomarkers selected from selected from Ephrin B2, Epidermal Growth Factor, Ferritin-heavy chain, Apotransferrin, Phosphoinositide 3-kinase, Mdm2-binding protein isoform X3, Tubulin polyglutamylase TTLL13 isoform X7, Heparin sulfate glucosamine 3-O-sulfotransferase 2, serotransferrin, alpha-2-macroglobulin, immunoglobulin lambda-like polypeptide 5, ceruloplasmin, alpha-1-antitrypsin precursor, complement C3 preproprotein, coiled-coil domain-containing protein 144A, cytochrome P450 3A43, myosin phosphatase Rho-interacting protein, collagen alpha-6(IV) chain protein FAM110A, DNA polymerase epsilon catalytic subunit A, semaphorin-5B, ephrin type-B receptor 1, zinc finger protein 532, tyrosine-tRNA ligase (cytoplasmic), mismatch repair endonuclease PMS2, cadherin EGF LAG seven-pass G-type receptor, cytoskeleton-associated protein 2, tyrosine-tRNA ligase (cytoplasmic), zinc finger protein 618, cytoskeleton-associated protein 2, alpha-2-macroglobulin, serine/threonine-protein kinase OSR1, collagen alpha-1(XII) chain, zinc finger ZZ-type and EF-hand domain-containing protein 1, iporin, phospholipid-transporting ATPase IG, collagen alpha-6(IV) chain, spatacsin, short stature homeobox protein 2, collagen alpha-1(XII) chain, ubiquitin carboxyl-terminal hydrolase 28, phospholipid-transporting ATPase IG, cyclin-dependent kinase 13, A-kinase anchor protein 9, zinc finger protein 417, sorting nexin-13, hemoglobin subunit beta, hemoglobin subunit delta, hemoglobin subunit alpha, cytochrome P450 3A4 isoform, filamin-C, apolipoprotein A-IV, nebulin, SAA2-SAA2 protein precursor, plasminogen isoform 1 precursor, ATPase family AAA domain-containing protein 5, DNA damage-induced apoptosis suppressor protein, E3 ubiquitin-protein ligase Midline-1, mitogen-activated protein kinase kinase kinase kinase 1, mucin-16, probable E3 ubiquitin-protein ligase HECTD4, protein RRP5 homolog , retrotransposon Gag-like protein 9, serine/threonine-protein phosphatase 2A regulatory subunit B″ subunit gamma, and uncharacterized protein C12orf42 at a time point selected from one or more of before, during, or after administration of said potential therapeutic agent, wherein a change in a level of a biomarker as compared to a control value indicates that said potential therapeutic has efficacy in treating IPF in an individual in need thereof.

12. A method of diagnosing and treating a pulmonary disease in an individual in need thereof, comprising
a) establishing, using a computing system, a model for characterizing the disease state of an individual having or likely to have a pulmonary disease, by using expression level data of an one or more biomarkers selected from Ephrin B2, Epidermal Growth Factor, Ferritin-heavy chain, Apotransferrin, Phosphoinositide 3-kinase, Mdm2-binding protein isoform X3, Tubulin polyglutamylase TTLL13 isoform X7, Heparin sulfate glucosamine 3-O-sulfotransferase 2, serotransferrin, alpha-2-macroglobulin, immunoglobulin lambda-like polypeptide 5, ceruloplasmin, alpha-1-antitrypsin precursor, complement C3 preproprotein, coiled-coil domain-containing protein 144A, cytochrome P450 3A43, myosin phosphatase Rho-interacting protein, collagen alpha-6(IV) chain protein FAM110A, DNA polymerase epsilon catalytic subunit A, semaphorin-5B, ephrin type-B receptor 1, zinc finger protein 532, tyrosine-tRNA ligase (cytoplasmic), mismatch repair endonuclease PMS2, cadherin EGF LAG seven-pass G-type receptor, cytoskeleton-associated protein 2, tyrosine-tRNA ligase (cytoplasmic), zinc finger protein 618, cytoskeleton-associated protein 2, alpha-2-macroglobulin, serine/threonine-protein kinase OSR1, collagen alpha-1(XII) chain, zinc finger ZZ-type and EF-hand domain-containing protein 1, iporin, phospholipid-transporting ATPase IG, collagen alpha-6(IV) chain, spatacsin, short stature homeobox protein 2, collagen alpha-1(XII) chain, ubiquitin carboxyl-terminal hydrolase 28, phospholipid-transporting ATPase IG, cyclin-dependent kinase 13, A-kinase anchor protein 9, zinc finger protein 417, sorting nexin-13, hemoglobin subunit beta, hemoglobin subunit delta, hemoglobin subunit alpha, cytochrome P450 3A4 isoform, filamin-C, apolipoprotein A-IV, nebulin, SAA2-SAA2 protein precursor, plasminogen isoform 1 precursor, ATPase family AAA domain-containing protein 5, DNA damage-induced apoptosis suppressor protein, E3 ubiquitin-protein ligase Midline-1, mitogen-activated protein kinase kinase kinase kinase 1, mucin-16, probable E3 ubiquitin-protein ligase HECTD4, protein RRP5 homolog , retrotransposon Gag-like protein 9, serine/threonine-protein phosphatase 2A regulatory subunit B″ subunit gamma, and uncharacterized protein C12orf42.
b) contacting a biological sample from said individual with at least one detection agent capable of specifically binding to said one or more biomarkers;
c) acquiring an expression level from said one or more biomarkers measured from said biological sample;
d) characterizing said individual as one or more of likely to have a lung disease, having stable lung disease, having progressive lung disease, or having improving lung disease; and
e) optionally, administering a therapy to said individual based on said characterization.

13. The method of claim 12, wherein said disease is idiopathic pulmonary fibrosis (IPF).
